(19) United States
(12) Patent Application Publication
(10) Pub. No.: US 2011/0218235 A1
(43) Pub. Date: Sep. 8, 2011

(54) COMPOUND AND COMPOSITION AND THEIR USES THEREOF

(75) Inventor: MAHESH KANDULA, MEDAPADU (IN)

(73) Assignee: KRISHANI BIOSCIENCES (P) LTD, HYDERABAD (IN)

(21) Appl. No.: 13/031,420

(22) Filed: Feb. 21, 2011

Related U.S. Application Data
(60) Provisional application No. 61/310,719, filed on Mar. 5, 2010.

(51) Int. Cl. A61K 31/385; C07D 339/04; A61P 1/16

(52) U.S. Cl. 514/440; 549/39

(57) ABSTRACT

A compound, composition, method of synthesizing and using the compound of formula 1 are disclosed. The compound of formula I also comprises of salts, polymorphs, solvates, and hydrates thereof. The compound may be formulated as pharmaceutical compositions. The pharmaceutical compositions may be formulated for peroral, topical, transmucosal, inhalation, targeted delivery and sustained release formulations. Such compositions may be used to treat hepatic and genetic disorders related to copper overload.

### CROSS REFERENCE TO RELATED APPLICATION

This application claims priority to U.S. Provisional Patent Application No. 61/310,719, filed on Mar. 5, 2010. This application is hereby incorporated by this reference in their entireties for all of its teachings.

### TECHNICAL FIELD

This disclosure generally relates to compound and their synthesis. More particularly, this disclosure relates to treating mammals with pharmaceutically acceptable amount of compounds, composition and the prodrugs of the compound.

### BACKGROUND ART

Metal accumulation has been responsible for many dysfunctions in hepatic disorders. Pathophysiologic mechanisms responsible for cerebral dysfunction and neuronal cell death in hepatocerebral disorders, such as Wilson's Disease, post-shunt myelopathy, hepatic encephalopathy, and acquired non-Wilsonian hepatocerebral degeneration are a major feature of hepatocerebral disorders. Morphologic changes to astrocytes (Alzheimer type II astrocytosis) include neurotoxic effects of metals such as copper, manganese, and iron. Management and treatment of hepatocerebral disorders include chelation therapy (Wilson's Disease) and liver transplantation among others.

Copper is found in all living organisms and is a crucial trace element in redox chemistry, growth and development. Overload or deficiency of copper is associated, respectively, with Wilson disease (WD) and Menkes disease (MD), which are of genetic origin. Researches on Menkes and Wilson disorders have provided useful insights in the field of copper homeostasis and in particular into the understanding of intracellular trafficking and distribution of copper at molecular levels. Therapies based on metal supplementation with copper histidine or removal of copper excess by means of specific copper chelators are currently effective in treating MD and WD, respectively. Copper chelation therapy is now attracting much attention for the investigation and treatment of various neurodegenerative disorders such as Alzheimer, Parkinson and Creutzfeldt-Jakob. An excess of copper appears to be an essential co-factor for angiogenesis. Moreover, elevated levels of copper have been found in many types of human cancers, including prostate, breast, colon, lung, and brain. On this basis, the employment of copper chelators has been reported to be of therapeutic value in the treatment of several types of cancers as anti-angiogenic molecules. There is a need for development of new copper chelator and an anticancer metallodrug with improved specificity and decreased toxic side effects.

### SUMMARY OF DISCLOSURE

In one embodiment, a compound comprising of Formula 1 (also mentioned as formula 1) is disclosed.

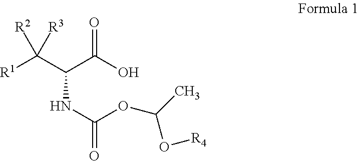

Another embodiment, a pharmaceutical composition comprising of one or more compounds of formula 1, an intermediate, a prodrug, pharmaceutical acceptable salt of compound formula 1 with one or more of pharmaceutically acceptable carriers, and vehicles or diluents are disclosed. These compositions may be used in the treatment of diseases related to copper retention and its complications in hepatic diseases and/or disorders.

In another embodiment, the present disclosure relates to the compound and composition of formula 1, or pharmaceutically acceptable salts thereof,

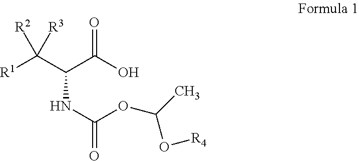

### Wherein,

R¹, R², and R³ each independently represents hydrogen, thiol, alkyl, alkyl thiol, acetyl thiol, disulfide, acyl, acylalkyl, alkenyl, alkylthioalkyl, alkynyl, alkoxyaryl, alkoxyalkyl, aryl, aralkyl, aryloxyalkyl, arylthioalkyl, cycloalkyl, ether, ester, heteroaryl, heterocyclyl, lower alkyl, sulfone, sulfoxide, or hydroxyalkyl;
R⁴ represents at least one of a residue of guanidine, a residue of hydrazine, an acid, a residue of pyruvic acid, a residue of oxaloacetic acid, a residue of tocopherol, a residue of ascorbic acid, a residue of thiamine, thioctic acid, a residue of thioctic acid, a residue of acetyl cysteine, a residue of alpha-keto glutaric acid, a residue of dimercaprol, a residue of an NO donor, a residue of glutathione and an analog of any one of the foregoing.

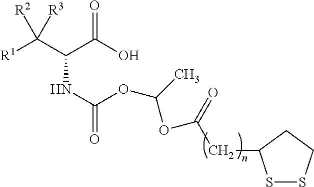

where, n represents an integer from 0 to 8;

In another preferred embodiment, formula 1 may represent the following compound:

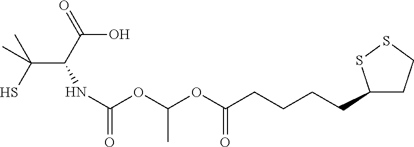

### Wherein:

R¹, R², and R³ each independently represents hydrogen, thiol, alkyl, alkyl thiol, acetyl thiol, disulfide, acyl, acylalkyl, alkenyl, alkylthioalkyl, alkynyl, alkoxyaryl, alkoxyalkyl, aryl, aralkyl, aryloxyalkyl, arylthioalkyl, cycloalkyl, ether, ester, heteroaryl, heterocyclyl, lower alkyl, sulfone, sulfoxide, or hydroxyalkyl;
R⁴ represents thioctic acid and where n represent the integer between 4 to 8.

In one embodiment, R¹, R² and R³ represents, hydrogen, methyl, ethyl or thiol and R⁴ represents R-isomer of residue or analog or derivative or metabolite of thioctic acid.

Furthermore, this disclosure provides an embodiment comprising a composition:

a) R-(+)-lipoic acid or Thioctic acid

b) Zinc acetate (or) Triethylene tetramine; and

c) a compound of Formula 1

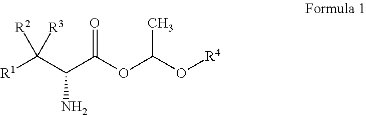

### Wherein,

R¹, R², and R³ each independently represents hydrogen, thiol, alkyl, alkyl thiol, acetyl thiol, disulfide, acyl, acylalkyl, alkenyl, alkylthioalkyl, alkynyl, alkoxyaryl, alkoxyalkyl, aryl, aralkyl, aryloxyalkyl, arylthioalkyl, cycloalkyl, ether, ester, heteroaryl, heterocyclyl, lower alkyl, sulfone, sulfoxide, or hydroxyalkyl;
R⁴ represents at least one of a residue of guanidine, a residue of hydrazine, an acid, a residue of pyruvic acid, a residue of oxaloacetic acid, a residue of tocopherol, a residue of ascorbic acid, a residue of thiamine, thioctic acid, a residue of thioctic acid, a residue of acetyl cysteine, a residue of alpha-keto glutaric acid, a residue of dimercaprol, a residue of an NO donor, a residue of glutathione and an analog of any one of the foregoing.

In one embodiment the therapeutically effective amount may be rendered, but not limited to, as an injection. Other embodiments may include peroral, topical, transmucosal, inhalation, targeted delivery and sustained release formulations. The topical application may be a ophthalmic drug used as drops, targeted delivery may be injection to the organ and peroral may be syrup, tablet or capsule.

Herein, the application additionally provides kits comprising the pharmaceutical compositions described herein. The kits may further comprise instructions for use in the treatment of diseases related to copper retention, hepatic disorders or its related complications.

Furthermore, herein is provided a kit comprising a first composition and a second composition, wherein a) the first composition is R-(+)-lipoic acid; b) the second composition is a compound of Formula 1 and c) the third composition is triethylene tetramine (or) Zinc acetate or Ammonium tetrathiomolybdate:

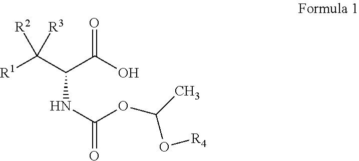

### Wherein,

R¹, R², and R³ each independently represents hydrogen, thiol, alkyl, alkyl thiol, acetyl thiol, disulfide, acyl, acylalkyl, alkenyl, alkylthioalkyl, alkynyl, alkoxyaryl, alkoxyalkyl, aryl, aralkyl, aryloxyalkyl, arylthioalkyl, cycloalkyl, ether, ester, heteroaryl, heterocyclyl, lower alkyl, sulfone, sulfoxide, or hydroxyalkyl;
R⁴ represents at least one of a residue of guanidine, a residue of hydrazine, an acid, a residue of pyruvic acid, a residue of oxaloacetic acid, a residue of tocopherol, a residue of ascorbic acid, a residue of thiamine, thioctic acid, a residue of thioctic acid, a residue of acetyl cysteine, a residue of alpha-keto glutaric acid, a residue of dimercaprol, a residue of an NO donor, a residue of glutathione and an analog of any of the foregoing.

Additionally, in another embodiment the instant application discloses several methods of synthesizing the composition of formula I.

In another embodiment, R-lipoic acid, Dimercaprol, Zinc acetate, Ammonium tetrathiomolybdate or triethylene tetramine is combined with a pharmaceutically acceptable salt of the compound of formula 1.

The compound, composition, method of synthesis, and treatment disclosed herein may be implemented in any means for achieving various aspects, and may be executed in a form suitable for the mammal. Other features will be apparent from the accompanying detailed description that follows.

### BRIEF DESCRIPTION OF DRAWINGS

FIG. 1 shows a first method of synthesis of compound representing formula 1.

FIG. 2 shows a second method of synthesis of compound represented by formula 1.

### DETAILED DESCRIPTION

In the present disclosure metal chelating compounds and compositions are disclosed. The compound comprises of formula 1. Furthermore, the composition comprises of R-lipoic acid, Dimercaprol, Zinc acetate, Ammonium tetrathiomolybdate or triethylene tetramine is combined with a pharmaceutically acceptable salt of the compound of formula 1. In another embodiment, methods of making the formula 1 are disclosed.

The compound may also comprise of tartrate, esylate, mesylate, sulfate salts and hydrate salt of formula 1. Herein the application also provides a kit comprising any of the pharmaceutical compositions disclosed herein. The kit may comprise instructions for use in the treatment of diseases associated to copper toxicity, hepatic disorders or related complications.

### DEFINITIONS

As used herein, the following terms and phrases shall have the meanings set forth below. Unless defined otherwise, all technical and scientific terms used herein have the same meaning as commonly understood to one of ordinary skill in the art.

The term “alkyl” refers to the radical of saturated aliphatic groups, including straight-chain alkyl groups, branched-chain alkyl groups, cycloalkyl (alicyclic) groups, alkyl-substituted cycloalkyl groups, and cycloalkyl-substituted alkyl groups. In preferred embodiments, a straight chain or branched chain alkyl has 30 or fewer carbon atoms in its backbone (e.g., C₁-C₃₀ for straight chains, C₃-C₃₀ for branched chains), and more preferably 20 or fewer. Likewise, preferred cycloalkyls have from 3-10 carbon atoms in their ring structure, and more preferably have 5, 6 or 7 carbons in the ring structure.

The term “alkyl” as used herein refers to a saturated linear or branched-chain monovalent hydrocarbon radical of one to twelve carbon atoms. Examples of alkyl groups include, but are not limited to, methyl (Me, —CH₃), ethyl (Et, —CH₂CH₃), 1-propyl (n-Pr, n-propyl, —CH₂CH₂CH₃), 2-propyl (i-Pr, i-propyl, —CH(CH₃)₂), 1-butyl (n-Bu, n-butyl, —CH₂CH₂CH₂CH₃), 2-methyl-1-propyl (i-Bu, i-butyl, —CH₂CH(CH₃)₂), 2-butyl (s-Bu, s-butyl, —CH(CH₃)CH₂CH₃), 2-methyl-2-propyl (t-Bu, t-butyl, —C(CH₃)₃), 1-pentyl (n-pentyl, —CH₂CH₂CH₂CH₂CH₃), 2-pentyl (—CH(CH₃)CH₂CH₂CH₃), 3-pentyl (—CH(CH₂CH₃)₂), 2-methyl-2-butyl (—C(CH₃)₂CH₂CH₃), 3-methyl-2-butyl (—CH(CH₃)CH(CH₃)₂), 3-methyl-1-butyl (—CH₂CH₂CH(CH₃)₂), 2-methyl-1-butyl (—CH₂CH(CH₃)CH₂CH₃), 1-hexyl (—CH₂CH₂CH₂CH₂CH₂CH₃), 2-hexyl (—CH(CH₃)CH₂CH₂CH₂ CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), 3,3-dimethyl-2-butyl (—CH(CH₃)C(CH₃)₃, 1-heptyl, 1-octyl, and the like. [0014] The term “alkenyl” refers to linear or branched-chain monovalent hydrocarbon radical of two to twelve carbon atoms with at least one site of unsaturation, i.e., a carbon-carbon, sp double bond, wherein the alkenyl radical includes radicals having “cis” and “trans” orientations, or alternatively, “E” and “Z” orientations. Examples include, but are not limited to, ethylenyl or vinyl (—CH═CH₂), allyl (—CH₂CH═CH₂), and the like. The term “alkynyl” refers to a linear or branched monovalent hydrocarbon radical of two to twelve carbon atoms with at least one site of unsaturation, i.e., a carbon-carbon, sp triple bond. Examples include, but are not limited to, ethynyl (—C≡CH), propynyl (propargyl, —CH₂C≡CH), and the like.

Moreover, the term “alkyl” (or “lower alkyl”) as used throughout the specification, examples, and claims is intended to include both “unsubstituted alkyls” and “substituted alkyls”, the latter of which refers to alkyl moieties having substituents replacing a hydrogen on one or more carbons of the hydrocarbon backbone. Such substituents, if not otherwise specified, may include, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain may themselves be substituted, if appropriate. For instance, the substituents of a substituted alkyl may include substituted and unsubstituted forms of amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF₃, —CN and the like. Exemplary substituted alkyls are described below. Cycloalkyls may be further substituted with alkyls, alkenyls, alkoxys, alkylthios, aminoalkyls, carbonyl-substituted alkyls, —CF₃, —CN, and the like.

The term “acyl” is art-recognized and refers to a group represented by the general formula hydrocarbylC(O)—, preferably alkylC(O)—.

“Aryl” means a monocyclic or polycyclic ring assembly wherein each ring is aromatic or when fused with one or more rings forms an aromatic ring assembly. If one or more ring atoms is not carbon (e.g., N, S), the aryl is a heteroaryl. Cₓ aryl and Cₓ_{^{-Y}} aryl are typically used where X and Y indicate the number of carbon atoms in the ring.

The term “acylamino” is art-recognized and refers to an amino group substituted with an acyl group and may be represented, for example, by the formula hydrocarbyl C(O)NH—.

The term “acylalkyl” is art-recognized and refers to an alkyl group substituted with an acyl group and may be represented, for example, by the formula hydrocarbyl C(O)alkyl.

The term “acyloxy” is art-recognized and refers to a group represented by the general formula hydrocarbylC(O)O—, preferably alkylC(O)O—.

The term “alkoxy” refers to an alkyl group, preferably a lower alkyl group, having an oxygen attached thereto. Representative alkoxy groups include methoxy, ethoxy, propoxy, tert-butoxy and the like.

The term “alkoxyalkyl” refers to an alkyl group substituted with an alkoxy group and may be represented by the general formula alkyl-O-alkyl.

The term “alkenyl”, as used herein, refers to an aliphatic group containing at least one double bond and is intended to include both “unsubstituted alkenyls” and “substituted alkenyls”, the latter of which refers to alkenyl moieties having substituents replacing a hydrogen on one or more carbons of the alkenyl group. Such substituents may occur on one or more carbons that are included or not included in one or more double bonds.

Moreover, such substituents include all those contemplated for alkyl groups, as discussed below, except where stability is prohibitive. For example, substitution of alkenyl groups by one or more alkyl, carbocyclyl, aryl, heterocyclyl, or heteroaryl groups is contemplated.

The term “alkylamino”, as used herein, refers to an amino group substituted with at least one alkyl group.

The term “alkylthio”, as used herein, refers to a thiol group substituted with an alkyl group and may be represented by the general formula alkylS—.

The term “alkynyl”, as used herein, refers to an aliphatic group containing at least one triple bond and is intended to include both “unsubstituted alkynyls” and “substituted alkynyls”, the latter of which refers to alkynyl moieties having substituents replacing a hydrogen on one or more carbons of the alkynyl group. Such substituents may occur on one or more carbons that are included or not included in one or more triple bonds. Moreover, such substituents include all those contemplated for alkyl groups, as discussed above, except where stability is prohibitive. For example, substitution of alkynyl groups by one or more alkyl, carbocyclyl, aryl, heterocyclyl, or heteroaryl groups is contemplated.

The term “ether”, as used herein, refers to a hydrocarbyl group linked through an oxygen to another hydrocarbyl group. Accordingly, an ether substituent of a hydrocarbyl group may be hydrocarbyl-O—. Ethers may be either symmetrical or unsymmetrical. Examples of ethers include, but are not limited to, heterocycle-O-heterocycle and aryl-O-heterocycle. Ethers include “alkoxyalkyl” groups, which may be represented by the general formula alkyl-O-alkyl.

The terms “halo” and “halogen” as used herein means halogen and includes chloro, fluoro, bromo, and iodo.

The terms “hetaralkyl” and “heteroaralkyl”, as used herein, refers to an alkyl group substituted with a hetaryl group.

The term “heteroalkyl”, as used herein, refers to a saturated or unsaturated chain of carbon atoms and at least one heteroatom, wherein no two heteroatoms are adjacent.

The terms “heteroaryl” and “hetaryl” include substituted or unsubstituted aromatic single ring structures, preferably 5- to 7-membered rings, more preferably 5- to 6-membered rings, whose ring structures include at least one heteroatom, preferably one to four heteroatoms, more preferably one or two heteroatoms. The terms “heteroaryl” and “hetaryl” also include polycyclic ring systems having two or more cyclic rings in which two or more carbons are common to two adjoining rings wherein at least one of the rings is heteroaromatic, e.g., the other cyclic rings may be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, pyrazole, pyridine, pyrazine, pyridazine, and pyrimidine, and the like.

The term “heteroatom” as used herein means an atom of any element other than carbon or hydrogen. Preferred heteroatoms are nitrogen, oxygen, and sulfur.

The terms “heterocyclyl”, “heterocycle”, and “heterocyclic” refer to substituted or unsubstituted non-aromatic ring structures, preferably 3- to 10-membered rings, more preferably 3- to 7-membered rings, whose ring structures include at least one heteroatom, preferably one to four heteroatoms, more preferably one or two heteroatoms. The terms “heterocyclyl” and “heterocyclic” also include polycyclic ring systems having two or more cyclic rings in which two or more carbons are common to two adjoining rings wherein at least one of the rings is heterocyclic, e.g., the other cyclic rings may be cycloalkyls, cycloalkenyls, cycloalkynyls, aryls, heteroaryls, and/or heterocyclyls. Heterocyclyl groups include, for example, piperidine, piperazine, pyrrolidine, morpholine, lactones, lactams, and the like.

The term “heterocyclylalkyl”, as used herein, refers to an alkyl group substituted with a heterocycle group.

The term “hydrocarbyl”, as used herein, refers to a group that is bonded through a carbon atom that does not have a ═O or ═S substituent, and typically has at least one carbon-hydrogen bond and a primarily carbon backbone, but may optionally include heteroatoms. Thus, groups like methyl, ethoxyethyl, 2-pyridyl, and trifluoromethyl are considered to be hydrocarbyl for the purposes of this application, but substituents such as acetyl (which has a ═O substituent on the linking carbon) and ethoxy (which is linked through oxygen, not carbon) are not. Hydrocarbyl groups include, but are not limited to aryl, heteroaryl, carbocycle, heterocycle, alkyl, alkenyl, alkynyl, and combinations thereof.

The term “hydroxyalkyl”, as used herein, refers to an alkyl group substituted with a hydroxy group.

The term “ketone” is art-recognized and may be represented, for example, by the formula C(O)R₉, wherein R₉ represents a hydrocarbyl group.

The term “lower” when used in conjunction with a chemical moiety, such as, acyl, acyloxy, alkyl, alkenyl, alkynyl, or alkoxy is meant to include groups where there are ten or fewer non-hydrogen atoms in the substituent, preferably six or fewer. A “lower alkyl”, for example, refers to an alkyl group that contains ten or fewer carbon atoms, preferably six or fewer. Lower alkyls include methyl and ethyl. In certain embodiments, acyl, acyloxy, alkyl, alkenyl, alkynyl, or alkoxy substituents defined herein are respectively lower acyl, lower acyloxy, lower alkyl, lower alkenyl, lower alkynyl, or lower alkoxy, whether they appear alone or in combination with other substituents, such as in the recitations hydroxyalkyl and aralkyl (in which case, for example, the atoms within the aryl group are not counted when counting the carbon atoms in the alkyl substituent).

The term “substituted” refers to moieties having substituents replacing hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents may be one or more and the same or different for appropriate organic compounds. For purposes of this application, the heteroatoms such as nitrogen may have hydrogen substituents and/or any permissible substituents of organic compounds described herein which satisfy the valences of the heteroatoms. Substituents may include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain may themselves be substituted, if appropriate.

Unless specifically stated as “unsubstituted,” references to chemical moieties herein are understood to include substituted variants. For example, reference to an “aryl” group or moiety implicitly includes both substituted and unsubstituted variants.

“Substituted or unsubstituted” means that a given moiety may consist of only hydrogen substituents through available valencies (unsubstituted) or may further comprise one or more non-hydrogen substituents through available valencies (substituted) that are not otherwise specified by the name of the given moiety. For example, isopropyl is an example of an ethylene moiety that is substituted by —CH₃. In general, a non-hydrogen substituent may be any substituent that may be bound to an atom of the given moiety that is specified to be substituted. Examples of substituents include, but are not limited to, aldehyde, alicyclic, aliphatic, (C₁₋₁₀) alkyl, alkylene, alkylidene, amide, amino, aminoalkyl, aromatic, aryl, bicycloalkyl, bicycloaryl, carbamoyl, carbocyclyl, carboxyl, carbonyl group, cycloalkyl, cycloalkylene, ester, halo, heterobicycloalkyl, heterocycloalkylene, heteroaryl, heterobicycloaryl, heterocycloalkyl, oxo, hydroxy, iminoketone, ketone, nitro, oxaalkyl and oxoalkyl moieties, each of which may optionally also be substituted or unsubstituted. In one particular embodiment, examples of substituents include, but are not limited to, hydrogen, halo, nitro, cyano, thio, oxy, hydroxy, carbonyloxy, (C₁₋₁₀) alkoxy, (C₄₋₁₂) aryloxy, hetero (C₁₋₁₀)aryloxy, carbonyl, oxycarbonyl, aminocarbonyl, amino, (C₁₋₁₀) alkylamino, sulfonamido, imino, sulfonyl, sulfinyl, (C1-10)alkyl, halo (C1-10) alkyl, hydroxy (C1-10) alkyl, carbonyl (C1-10)alkyl, thiocarbonyl (C1-10)alkyl, sulfonyl (C1-10) alkyl, sulfinyl (C1-10) alkyl, (C1-10)azaalkyl, imino (C1-10) alkyl, (C₃₋₁₂) cycloalkyl (C1-₅) alkyl, hetero (C3-12) cycloalkyl (Ci-I₀) alkyl, aryl (Ci-I₀) alkyl, hetero (C1-10) aryl (C1-5) alkyl, (C₉₋₁₂) bicycloaryl (Ci_s) alkyl, hetero (Ce-₁₂) bicycloaryl (Ci_{—5}) alkyl, (C3-12) cycloalkyl, hetero (C3-12) cycloalkyl, (C9-12) bicycloalkyl, hetero (C₃₋₁₂) bicycloalkyl, (C₄₋₁₂) aryl, hetero (C1-10) aryl, (C₉₋₁₂) bicycloaryl and hetero (C₄₋₁₂) bicycloaryl. In addition, the substituent is itself optionally substituted by a further substituent. In one particular embodiment, examples of the further substituent include, but are not limited to, hydrogen, halo, nitro, cyano, thio, oxy, hydroxy, carbonyloxy, (C1-10)alkoxy, (C₄—I₂) aryloxy, hetero (C1-10) aryloxy, carbonyl, oxycarbonyl, aminocarbonyl, amino, (C1-10) alkylamino, sulfonamido, imino, sulfonyl, sulfinyl, (C1-10) alkyl, halo (C1-10)alkyl, hydroxy (C1-10) alkyl, carbonyl (C1-10) alkyl, thiocarbonyl (C1-10) alkyl, sulfonyl (C1-10) alkyl, sulfinyl (C1-10)alkyl, (C1-10) azaalkyl, imino (C1-10) alkyl, (C₃₋₁₂) cycloalkyl (C₁₋₅) alkyl, hetero (C3-12) cycloalkyl (C1-10) alkyl, aryl (C₁_{_{—}}₁₀) alkyl, hetero (Ci-io) aryl (Ci_{—5}) alkyl, (C₉-I₂) bicycloaryl (C₁₋₅) alkyl, hetero (C₈₋₁₂) bicycloaryl (Ci_s) alkyl, (C3-12) cycloalkyl, hetero (C₃_{_{—}}12) cycloalkyl, (C₉₋₁₂) bicycloalkyl, hetero (C₃₋₁₂) bicycloalkyl, (C₄₋₁₂) aryl, hetero (C1-10) aryl, (C₉₋₁₂) bicycloaryl and hetero (C₄₋₁₂) bicycloaryl.

The compounds of the present compound of formula 1 may be present in the form of pharmaceutically acceptable salts. The compounds of the present disclosure may also be present in the form of pharmaceutically acceptable esters (i.e., the methyl and ethyl esters of the acids of formula I to be used as prodrugs). The compounds of the present disclosure may also be solvated, i.e. hydrated. The solvation may be effected in the course of the manufacturing process or may take place i.e. as a consequence of hygroscopic properties of an initially anhydrous compound of formula I (hydration).

Compounds that have the same molecular formula but differ in the nature or sequence of bonding of their atoms or the arrangement of their atoms in space are termed “isomers.” Isomers that differ in the arrangement of their atoms in space are termed “stereoisomers.” Diastereomers are stereoisomers with opposite configuration at one or more chiral centers which are not enantiomers. Stereoisomers bearing one or more asymmetric centers that are non-superimposable mirror images of each other are termed “enantiomers.” When a compound has an asymmetric center, for example, if a carbon atom is bonded to four different groups, a pair of enantiomers is possible. An enantiomer may be characterized by the absolute configuration of its asymmetric center or centers and is described by the R- and S-sequencing rules of Cahn, Ingold and Prelog, or by the manner in which the molecule rotates the plane of polarized light and designated as dextrorotatory or levorotatory (i.e., as (+) or (−)-isomers respectively). A chiral compound may exist as either individual enantiomer or as a mixture thereof. A mixture containing equal proportions of the enantiomers is called a “racemic mixture”.

The term “sulfate” is art-recognized and refers to the group OSO₃H, or a pharmaceutically acceptable salt thereof. A sulfate of compound of formula 1 or crystal thereof may be a hydrate. The number of the combined water can be controlled by varying the condition of recrystallization or drying. The salt form may be hydrochloride salt as well.

The term “polymorph” as used herein is art-recognized and refers to one crystal structure of a given compound.

“Residue” is an art-recognized term that refers to a portion of a molecule. For instance, a residue of thioctic acid may be: dihydrolipoic acid, bisnorlipoic acid, tetranorlipoic acid, 6,8-bismethylmercapto-octanoic acid, 4,6-bismethylmercapto-hexanoic acid, 2,4-bismethylmeracapto-butanoic acid, 4,6-bismethylmercapto-hexanoic acid.

The term “prodrug” is intended to encompass compounds that, under physiological conditions, are converted into the therapeutically active agents of the present disclosure. A common method for making a prodrug is to include selected moieties that are hydrolyzed under physiological conditions to reveal the desired molecule. In other embodiments, the prodrug is converted by an enzymatic activity of the host animal.

The term “prophylactic or therapeutic” treatment is art-recognized and includes administration to the host of one or more of the subject compositions. If it is administered prior to clinical manifestation of the unwanted condition (e.g., disease or other unwanted state of the host animal) then the treatment is prophylactic, i.e., it protects the host against developing the unwanted condition, whereas if it is administered after manifestation of the unwanted condition, the treatment is therapeutic, (i.e., it is intended to diminish, ameliorate, or stabilize the existing unwanted condition or side effects thereof).

The term “solvate” as used herein, refers to a compound formed by solvation (e.g., a compound formed by the combination of solvent molecules with molecules or ions of the solute).

The present disclosure also contemplates prodrugs of the compositions disclosed herein, as well as pharmaceutically acceptable salts of said prodrugs.

This application also discloses a pharmaceutical composition comprising a pharmaceutically acceptable carrier and the composition of thioctic acid or a residue of thioctic acid, dimercaprol or acetylcyteine and salts of a compound of Formula I or II. This application further discloses a pharmaceutical composition comprising a pharmaceutically acceptable carrier and (a) lipoic acid or residue of lipoate and (b) a compound of Formula I (c) dimercaprol or acetylcysteine or zinc acetate or ammonium thiomolybdate. The pharmaceutical composition may be formulated for systemic or topical administration. The pharmaceutical composition may be formulated for oral administration, injection, subdermal administration, or transdermal administration. The pharmaceutical composition may further comprise at least one of a pharmaceutically acceptable stabilizer, diluent, surfactant, filler, binder, and lubrimayt.

Additionally, the optimal concentration and/or quantities or amounts of any particular compound of formula I or composition may be adjusted to accommodate variations in the treatment parameters. Such treatment parameters include the clinical use to which the preparation is put, e.g., the site treated, the type of patient, e.g., human or non-human, adult or child, and the nature of the disease or condition.

Wilson's disease (WD) is an autosomal recessive disorder of the copper metabolism leading to the accumulation of this metal in different organs and tissues. Hepatic and neurological symptoms are the main clinical features of the disease. Copper-associated diseases are increasingly being reported in both man and animals. Copper also has a role in fatal, non-Wilson's liver diseases affecting young children with a genetic abnormality of copper metabolism. Excess accumulation of copper also occurs as a consequence of chronic liver diseases such as primary biliary cirrhosis, and chronic hepatitis in mammal such as humans and animals.

In certain embodiments, the compounds of formula I and compositions herein may be used to treat one or more copper toxicity related diseases or complications. Complications include Hepatic (cirrhosis, chronic active hepatitis, fulminant hepatic failure), Neurologic (bradykinesia, rigidity, tremor, ataxia, dyskinesia, dysarthria, seizures), Psychiatric (behavioral disturbances, cognitive impairment, psychosis), Orthalmologic (kayser-Fleischer rings, sunflow cataracts), Hematologic (haemolysis, coagulopathy), Renal (renal tubular defects, diminished glomerular filtration, nephrolithiasis), Cardiovascular (cardiomyopathy, arrhythmias, conduction disturbances, autonomic dysfunction), Musculoskeletal (osteomalacia, osteoporosis, degenerative joint diseases), Gastrointestinal (cholelithiasis, pancreatitis, bacterial peritonitis), Endocrinologic (amenorrhoea, spontaneous abortion, delayed puberty, gynecomastia), Dermatologic (hyperpigmentation, amaythosis nigrimays).

### Methods of Synthesis

### Example Synthesis 1

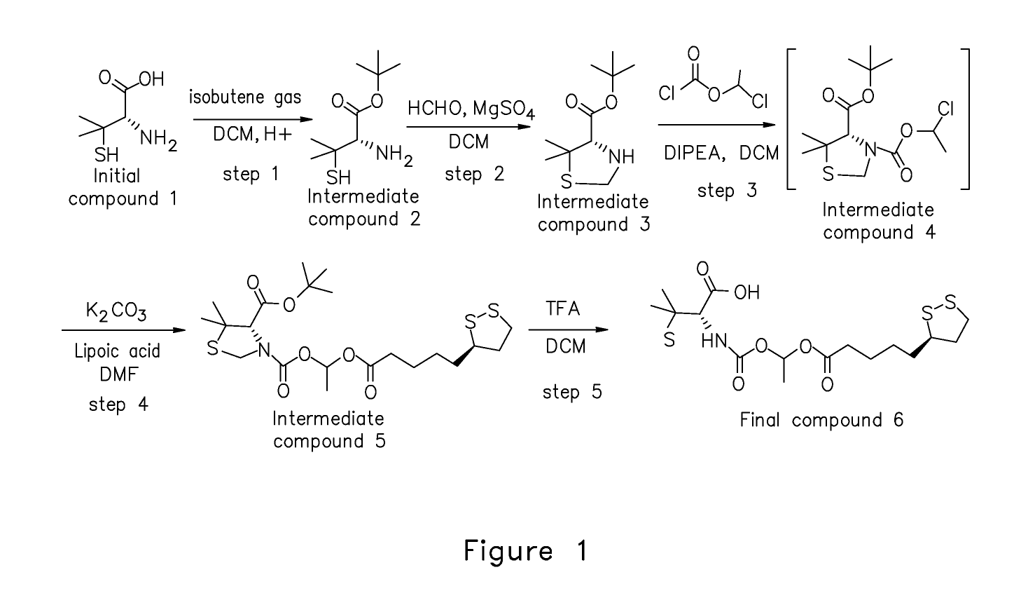

FIG. 1 shows a five step synthesis process for the composition of formula 1.

Step 1: (2S)-2-amino-3-methyl-3-sulfanyl-butanoic acid (initial compound 1) and Dichloromethane (DCM) were mixed together as a reaction mixture in a pressure bottle containing a magnetic stirrer. The pressure bottle containing the reaction mixture (intermediate compound 1) was securely closed with a rubber septum. The pressure bottle containing the reaction mixture was further cooled in 2-isoproponol/dry ice at 7-8° C. in the dry ice bath. Condensed isobutylene was transferred to the pressure bottle, using a cannula, followed by adding a few drops of sulfuric acid to the reaction mixture. The addition of isobutylene was continued for a period of 2 hours. Stirring of the reaction mixture was continued at room temperature for an additional 16 hours. The pressure bottle was kept in i-PrOH/dry ice bath and rubber septum was carefully removed. The reaction mixture was allowed to degas fully by stirring for several minutes. Saturated aqueous NaHCO3 was added to the reaction mixture, and the resultant reaction mixture was stirred for 2 hours at room temperature. The pH of the aqueous layer was measured and recorded as pH 8. Water was added for the removal of the emulsion that was formed during the neutralization step. The aqueous layer was treated using with DCM and then extracted. The entire DCM extracts were pooled together. The pooled DCM extracts were washed with saturated aqueous NaHCO3, water, and saturated aqueous NaCl solution. The resultant organic layer was dried in under MgSO4 atmosphere, concentrated and filtered under reduced pressure to yield intermediate compound 2.
Step 2: The condensation of amino thiol with paraformaldehyde in ethanol at room temperature for 30 minutes yielded thiazolidine derivative as intermediate compound 3.
Step 3: Thiazolidine derivative intermediate compound 3 was treated with 1.0 equivalents of 1-chloroethylchloroformate in presence of 1.5 equivalents of N,N-Diisopropylethylamine (DIPEA) in anhydrous dimercaprol at 0° C. The reaction mixture was allowed to stir for 30 min at 0° C. and yielded intermediate compound 4. On completion of the reaction the quality was monitored and recorded by performing thin layer chromatography (TLC). Based on the observation if the quality was satisfactory the intermediate compound 4 of step 3 was then directly used for the next step, without any further purification process.
Step 4: Potassium salt of Lipoic acid was obtained from reacting lipoic acid and anhydrous K₂CO₃ under dry Dimethylformaldehyde at 0° C. This reaction mixture of step 3 was added slowly into the above solution and then the crude reaction mixture was allowed to stir for 16 h at room temperature. Reaction was monitored by TLC. The crude reaction mixture was then vacuum distilled and fractionated using water and dichloromethane. The combined aqueous and organic layers were washed with brine solution, dried over anhydrous Na₂SO₄ and evaporated under reduced pressure. The crude reaction mixture was purified by column chromatography over 100-200 mesh silica gel to yield Lipoic acid derivative intermediate compound 5.
Step 5: Intermediate compound 5 obtained in the previous step 4 was treated with 25% trifluoracetic acid dissolved in DCM to hydrolyse the tert-butyl ester with the thiazolidine group of intermediate compound 5. This reaction yielded the final compound 6.

### Results of Synthesis 1

### Initial Compound 1

### (S)-2-amino-3-mercapto-3-methylbutanoic acid

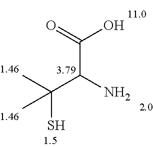

M.F: C5H11NO2S, Mol. Wt.: 149

TABLE 1
CHN Analysis:

| Atom | Intensity |
|---|---|
| C | 40.25 |
| H | 7.43 |
| N | 9.39 |
| O | 21.45 |
| S | 21.49 |

TABLE 2
H NMR Analysis

| δ | Protons | Group |
|---|---|---|
| 1.46 | 6H | 2 × CH3 |
| 3.79 | 1H | CH |

### Intermediate Compound 2

### (S)-tert-butyl 2-amino-3-mercapto-3-methylbutanoate

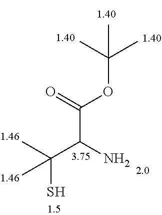

M.F: CH19NO2S, Mol. Wt.: 205

TABLE 3
CHN Analysis

| Atom | Intensity |
|---|---|
| C | 52.65 |
| H | 9.33 |
| N | 6.82 |
| O | 15.59 |
| S | 15.62 |

TABLE 4
H NMR Analysis

| δ | Protons | Group |
|---|---|---|
| 1.40 | 9H | 3 × CH3 |
| | | (tBu) |
| 1.46 | 6H | 2 × CH3 |
| 3.75 | 1H | CH |

### Intermediate Compound 3

### (S)-tert-butyl 5,5-dimethylthiazolidine-4-carboxylate

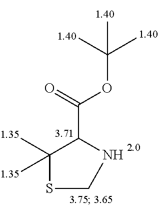

M.F: C10H19NO2S, Mol. Wt.: 217

TABLE 5
CHN Analysis

| Atom | Intensity |
|---|---|
| C | 55.27 |
| H | 8.81 |
| N | 6.44 |
| O | 14.72 |
| S | 14.75 |

TABLE 6
H NMR Analysis

| δ | Protons | Group |
|---|---|---|
| 1.40 | 9H | 3 × CH3 |
| | | (tBu) |
| 1.46 | 6H | 2 × CH3 |
| 3.65 | 2H | CH2 |
| 3.71 | 1H | CH |

### Intermediate Compound 5

### (4S)-3-(1-(5-((R)-1,2-dithiolan-3-yl)pentanoyloxy)ethyl)4-tert-butyldimethylthiazolidine-3,4-dicarboxylate

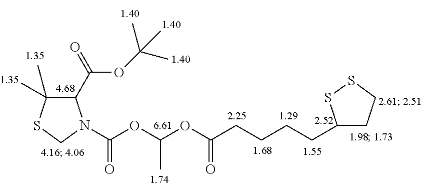

M.F: C21H35NO6S3, Mol. Wt.: 494

TABLE 7
CHN Analysis

| Atom | Intensity |
|---|---|
| C | 51.09 |
| H | 7.15 |
| N | 2.84 |
| O | 19.44 |
| S | 19.48 |

TABLE 8
H NMR Analysis

| δ | Protons | Group |
|---|---|---|
| 1.35 | 6H | 2 × CH₃ |
| 1.40 | 9H | 3 × CH₃ |
| | | (tBu) |
| 1.29, 1.55, 1.68, 1.98, 2.25 | 10H | 5 × CH₂ |
| 1.74 | 3H | CH₃ |
| 2.51-2.61 | 3H | SCH, |
| | | SCH₂ |
| 4.16 | 2H | SCH₂N |
| 4.68 | 1H | CH |
| 6.61 | 1H | OCHO |

### Final Compound 6

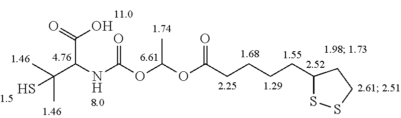

M.F: C16H27NO6S3, Mol. Wt.: 426

TABLE 9
CHN Analysis

| Atom | Intensity |
|---|---|
| C | 45.15 |
| H | 6.39 |
| N | 3.29 |
| O | 22.56 |
| S | 22.60 |

TABLE 10
H NMR Analysis

| δ | Protons | Group |
|---|---|---|
| 1.46 | 6H | 2 × CH₃ |
| 1.29, 1.55, 1.68, 1.98, 2.25 | 10H | 5 × CH₂ |
| 1.74 | 3H | CH₃ |
| 2.51-2.61 | 3H | SCH, |
| | | SCH₂ |
| 4.76 | 2H | SCH₂N |
| 4.68 | 1H | CH |
| 6.61 | 1H | OCHO |

### Example Synthesis 2

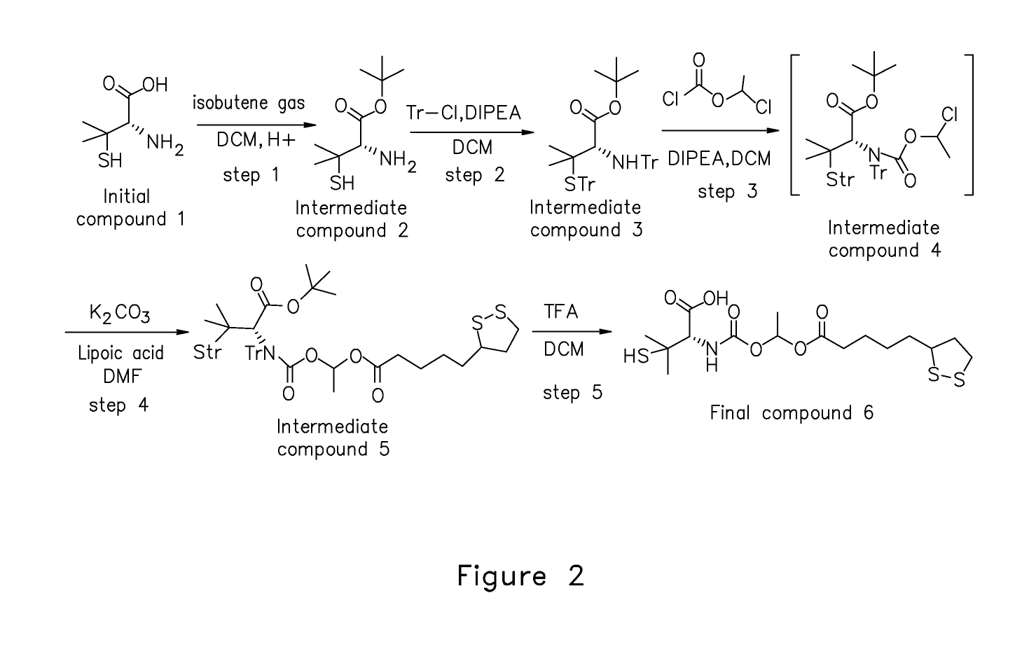

In synthesis 2, as shown in FIG. 2, in this approach protection of aminothiol derivative at producing intermediate compound 3 is different from the earlier synthesis 1, i.e., Trityl group is used instead of thiazolidine. The intermediate compound 2 is treated with 2.0 equivalent of trityl chloride in presence of diisopropylethylamine (DIPEA) dissolved in dichloromethane to yield a trityl derivative intermediate compound 3. The rest of the procedure remains the same.

In another embodiment, an effective dosage for the compound of Formula 1 is in the range of about 0.3 mg/kg/day to about 60 mg/kg/day in single or divided doses, for instance 1 mg/kg/day to about 50 mg/kg/day in single or divided doses. The compound of Formula 1 may be administered at a dose of, for example, less than 2 mg/kg/day, 5 mg/kg/day, 10 mg/kg/day, 20 mg/kg/day, 30 mg/kg/day, or 40 mg/kg/day. Compound of Formula 1 may also be administered to a human patient at a dose of, for example, between 50 mg and 1000 mg, between 100 mg and 800 mg, or less than 1000, 900, 800, 700, 600, 500, 400, 300, 200, or 100 mg per day. In certain embodiments, the compositions herein are administered at an amount that is less than 95%, 90%, 80%, 70%, 60%, 50%, 40%, 30%, 20%, or 10% of the compound of formula 1 is required for the same therapeutic benefit.

The present disclosure provides among other things compositions and methods for treating Copper toxicity related diseases and complications. While specific embodiments of the subject disclosure have been discussed, the above specification is illustrative and not restrictive. Many variations of the compounds, compositions and methods herein will become apparent to those skilled in the art upon review of this specification.

### INDUSTRIAL APPLICABILITY

There are multiple applications for compound of formula 1, composition of formula 1 with pharmaceutically acceptable additives to treat mammals suffering from hepatic diseases, more specifically genetic and abnormal accumulation of metal in the liver in general. These compositions may be used in the treatment of diseases related to copper retention and its complications in hepatic diseases.

What is claimed is:

1. A compound, comprising;
a pharmaceutically acceptable compound of formula 1: 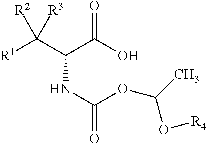
wherein R¹, R², and R³ each independently represents hydrogen, thiol, alkyl, alkyl thiol, acetyl thiol, disulfide, acyl, acylalkyl, alkenyl, alkylthioalkyl, alkynyl, alkoxyaryl, alkoxyalkyl, aryl, aralkyl, aryloxyalkyl, arylthioalkyl, cycloalkyl, ether, ester, heteroaryl, heterocyclyl, lower alkyl, sulfone, sulfoxide, or hydroxyalkyl; and
wherein R⁴ represents at least one of a residue of guanidine, a residue of hydrazine, an acid, a residue of pyruvic acid, a residue of oxaloacetic acid, a residue of tocopherol, a residue of ascorbic acid, a residue of thiamine, thioctic acid, a residue of thioctic acid, a residue of acetyl cysteine, a residue of alpha-keto glutaric acid, a residue of dimercaprol, a residue of an NO donor, a residue of glutathione and an analog of any one of the foregoing.

2. The compound of claim 1, further comprising:
a pharmaceutically acceptable compound of formula 1 comprising; 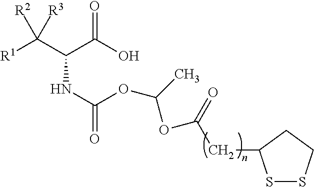
wherein: wherein, R¹, R², and R³ each independently represents hydrogen, thiol, alkyl, alkyl thiol, acetyl thiol, disulfide, acyl, acylalkyl, alkenyl, alkylthioalkyl, alkynyl, alkoxyaryl, alkoxyalkyl, aryl, aralkyl, aryloxyalkyl, arylthioalkyl, cycloalkyl, ether, ester, heteroaryl, heterocyclyl, lower alkyl, sulfone, sulfoxide, or hydroxyalkyl; and
R⁴ represents thioctic acid, wherein n is an integer that equals between 0 to 8.

3. A compound of claim 2, further comprising:
a pharmaceutically acceptable compound of formula 1 comprising; 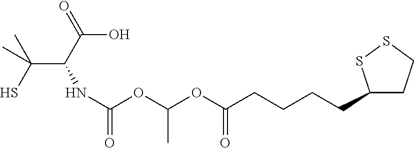
wherein, R¹, R², and R³ each independently represents hydrogen, thiol, alkyl, alkyl thiol, acetyl thiol, disulfide, acyl, acylalkyl, alkenyl, alkylthioalkyl, alkynyl, alkoxyaryl, alkoxyalkyl, aryl, aralkyl, aryloxyalkyl, arylthioalkyl, cycloalkyl, ether, ester, heteroaryl, heterocyclyl, lower alkyl, sulfone, sulfoxide, or hydroxyalkyl; and
wherein R⁴ is R-(+)-thioctic acid, wherein n is an integer that equals between 0 to 4.

4. The compound of claim 1, further comprising;
a pharmaceutically acceptable compound of formula 1 is at least one of a tartrate, esylate, mesylate, sulfate, hydrate and hydrochloride salt

5. A compound of claim 2, further comprising:
a composition to a mammal with a hepatic disorder comprising of compound represented by formula 1; and
wherein the composition comprises at least one of R-(+)-lipoic acid, acetylcysteine and dimercaprol and at least one of zinc acetate and triethylene tetramine.

6. The compound of claim 5, wherein administration is at least one of a peroral, topical, transmucosal, inhalation, targeted delivery and sustained release formulations.

7. A method of synthesis for a compound of formula 1, comprising:
mixing (2S)-2-amino-3-methyl-3-sulfanyl-butanoic acid and dimercaprol in a pressure bottle;
cooling the pressure bottle in dry ice and i-PrOH;
adding isobutylene and sulfuric acid for two hours;
stirring a resultant mixture for sixteen hours; and
degassing the resultant mixture in the pressure bottle at atmospheric pressure.

8. The method of synthesis of claim 7, further comprising;
adding sodium bi-carbonate to reduce the pH of the reaction mixture;
removing an emulsion that may have formed by adding water;
washing the reaction mixture with sodium bi-carbonate, water and saturated sodium chloride; and
filtering and drying the reaction mixture to obtain an intermediate compound 2.

9. The method of claim 8, further comprising;
performing condensation of intermediate compound 2 using paraformaldehyde to obtain an intermediate compound 3.

10. The method of claim 9, further comprising:
treating intermediate compound 2 with 2.0 equivalent of trityl chloride in presence of diisopropylethylamine dissolved in dichloromethane to yield a trityl derivative intermediate compound 3.

11. The method of claim 10, further comprising;
treating a thiazolidine derivative of intermediate compound 3 with 1-chloroethylchloroformate in presence of N,N-Diisopropylethylamine in anhydrous dimercaprol at 0° C.; and
stirring the reaction mixture 2 to obtain an intermediate compound 4.

12. The method of claim 11, wherein the ratio of 1-chloroethylchloroformate and N,N-diisopropylethylamine is 1:1.5.

13. The method of claim 12, further comprising:
testing the quality of intermediate compound 4 using thin layer chromatography.

14. The method of claim 13, further comprising:
reacting a lipoic acid and an anhydrous K₂CO₃ under dry dimethylformaldehyde at 0° C. to form a potassium salt of lipoic acid;
adding the intermediate compound 4 slowly to the potassium salt of lipoic acid;
stirring the mixture of potassium salt of lipoic acid and the intermediate compound 4 for 16 hours at room temperature; and
fractionating and vacuum distilling using water and dicholoromethnae to collect an aqueous layer and an organic layer.

15. The method of claim 14, further comprising:
washing the combined the aqueous layer and the organic layer with a brine solution;
drying the combined aqueous layer and organic layer over anhydrous sodium sulfate;
evaporating the combined aqueous layer and organic layer under reduced pressure to produce a crude reaction mixture; and
purifying the crude reaction mixture using column chromatography to yield an intermediate compound 5.

16. The method of claim 15, further comprising:
hydrolyzing the tert-butyl ester with a thiazolidne group of intermediate compound 5 using trifluoracetic acid dissolved in dimercaprol to yield the final compound 6.

17. A kit comprising a composition, comprising:
a) at least one of R-(+)-lipoic acid, acetylcysteine and dimercaprol;
b) at least one of zinc acetate and triethylene tetramine; and
c) a compound of Formula 1: 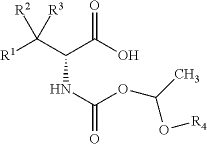
wherein R¹, R², and R³ each independently represents hydrogen, thiol, alkyl, alkyl thiol, acetyl thiol, disulfide, acyl, acylalkyl, alkenyl, alkylthioalkyl, alkynyl, alkoxyaryl, alkoxyalkyl, aryl, aralkyl, aryloxyalkyl, arylthioalkyl, cycloalkyl, ether, ester, heteroaryl, heterocyclyl, lower alkyl, sulfone, sulfoxide, or hydroxyalkyl; and
wherein R⁴ represents at least one of a residue of guanidine, a residue of hydrazine, an acid, a residue of pyruvic acid, a residue of oxaloacetic acid, a residue of tocopherol, a residue of ascorbic acid, a residue of thiamine, thioctic acid, a residue of thioctic acid, a residue of acetyl cysteine, a residue of alpha-keto glutaric acid, a residue of dimercaprol, a residue of an NO donor, a residue of glutathione and an analog of any one of the foregoing.

18. The kit of claim 17, further comprising instructions for use in the treatment of hepatic disorders and copper toxicity related diseases.

19. The kit of claim 18, further, comprising instructions for administering the composition to a mammal with the hepatic disorder comprising of compound represented by formula 1 and at least one of R-(+)-lipoic acid, acetylcysteine and dimercaprol; and at least one of zinc acetate and triethylene tetramine.
